(19) United States
(12) Patent Application Publication
(10) Pub. No.: US 2023/0127444 A1
(43) Pub. Date: Apr. 27, 2023

(54) COMPOSITION COMPRISING IRON OXIDE MAGNETIC PARTICLES FOR A TREATMENT OF LIVER CANCER

(71) Applicant: ZTI BIOSCIENCES CO, LTD., Seoul (KR)

(72) Inventors: Hyungseok CHANG, Suwon-si (KR); Yoon-Sik LEE, Anyang-si (KR); Ji Young RYU, Yongin-si (KR); Hyoungseok LEE, Anyang-si (KR); Yong-Sun PARK, Seoul (KR)

(73) Assignee: ZTI BIOSCIENCES CO, LTD., Seoul (KR)

(21) Appl. No.: 17/506,997

(22) Filed: Oct. 21, 2021

(30) Foreign Application Priority Data
Oct. 21, 2021 (KR) 10-2021-0140933

(51) Int. Cl. A61K 33/26; A61P 35/00; A61K 9/14

(52) U.S. Cl. CPC A61K 33/26; A61P 35/00; A61K 9/14

(57) ABSTRACT

Disclosed is a composition comprising iron oxide magnetic particles. The composition is delivered to a liver in a hepatocyte-specific manner to minimize damage to other organs, and is safely excreted from a body within a few weeks. Further, the particles have excellent hepatocyte targeting ability, and thus are used as a liver cancer treatment agent and a hepatocyte targeting carrier.

### CROSS-REFERENCE TO RELATED APPLICATION(S)

This application is based on and claims priority under 35 U.S.C. § 119 to Korean Patent Application No. 10-2021-0140933, filed Oct. 21, 2021 in the Korean Intellectual Property Office, the entire disclosure of which is incorporated by reference herein its entirety.

### BACKGROUND

### 1. Field

The disclosure relates to a composition comprising iron oxide magnetic particles, wherein the composition may be delivered specifically to hepatocytes and thus may be used as a liver cancer treatment agent and a hepatocyte targeting carrier.

### 2. Description of Related Art

Magnetic particles have been widely used in biomedical fields including cell labeling, magnetic resonance imaging (MRI), drug delivery, and hyperthermia. Among various types of magnetic particles, superparamagnetic iron oxide magnetic particles have been extensively studied in the biomedical field because of their high magnetic susceptibility and superparama.gnetism. Further, because magnetic particles generate heat when radiation or a magnetic field is applied thereto, the magnetic particles may be used for a contrast agent for magnetic resonance imaging (MRI) or a magnetic carrier for drug delivery in the field of nanomedicine or magnetic or radiation-based hyperthermia, and the like.

Iron oxide magnetic particles are mainly used as magnetic particles for hyperthermia. This is because the iron oxide magnetic particle is a material having an indirect band gap in which energy equal to an amount of momentum used is converted into heat which is released. Among the iron oxide magnetic particles, Fe₃O₄ (magnetite) or α-Fe (ferrite)-based magnetic particles have biocompatibility, heat induction ability, chemical stability, and unique magnetic properties. Because of these characteristics, research in which the iron oxide magnetic particles act as a magnetic heating element for hyperthermia is being actively conducted. Thus, the iron oxide magnetic particles as a magnetic heating element for hyperthermia has been approved for medical use by the US FDA. However, among the iron oxide magnetic particles, Fe₃O₄ particles are nano-sized and their crystal phase is easily changed to α-Fe₂O₃, γ-Fe₂O₃, etc. depending on conditions of an surrounding environment, and thus heat-generating properties and magnetic properties thereof change to reduce heat-generating ability thereof Research on MFe₂O₄ (M=Co, Ni, Mg) nanoparti cies based on Co, Ni, and Mg as another material is in progress. However, MFe₂O₄ (M=Co, Ni, Mg) nanoparticles may not be applied to an inside of a living body due to low exothermic temperature thereof.

Meanwhile, liver cancer is a malignant tumor originating from hepatocytes and is one of the cancers with a high incidence worldwide. In Korea, the liver caner has the fifth highest cancer incidence. However, the liver cancer has the second highest mortality rate next to a lung cancer, Korean has the highest liver cancer mortality rate among OECD countries.

Representative liver cancer treatments currently used clinically include targeting treating agents such as Bayer's Nexavar, Eisai's Lenvima, Bayer's Stivarga, Exelix's Cabometyx, Cyramza, etc. Between 2005 and 2018, Bayer's Nexavar was the only targeting treatment agent approved as a first-line treatment. However, Eisai's Lenvima approved in 2018 is currently known to be the most effective targeting treatment agent.

However, liver cancer has a high probability of resistance to drugs. In particular, when the liver cancer is treated with resection, embolization, or targeting therapy, a recurrence rate thereof is high, and a response rate thereto is also not high. Thus, the liver cancer is classified as a carcinoma with a low average survival rate. In addition, because most patients with liver cancer are accompanied by cirrhosis (80-90%), it is difficult to completely remove a cancer site. In addition, the liver cancer occurs multiple times and often invades blood vessels early. Thus, it is difficult to treat the liver cancer with a single therapy. The liver cancer has a high resistance to drugs, and a probability of recurrence thereof within 5 years is more than 90%, and thus recurrence and metastasis thereof are also high. Resection is primarily performed as a method of treating liver cancer. However, hepatic artery chemoembolization (TACE) is used as a representative treatment when resection is not possible. The TACE procedure is a non-surgical treatment for liver cancer that finds an artery that supplies nutrients to the liver tumor, administers an anticancer agent to the liver cancer cells, and then blocks the artery. Typically, liver embolization using Lipiodol has been most frequently applied clinically. However, there is a problem in that the anticancer agent dissolved in the water after the procedure does not accumulate in the liver cancer site and rapidly escapes into the systemic blood, so that sufficient anticancer effect cannot be obtained.

In radiopharmaceuticals BEXXAR®/Tositumoinab) approved by a certification organization including the FDA, due to separation of radioactive isotopes chemically bound to organic ligands in the body, side effects such as destruction of thyroid function occur. Thus, the radiopharmaceuticals may not be used as a therapeutic agent. On the other hand, iron oxide as a magnetic material causes toxicity in the body due to its high accumulation rate in body organs and poor excretion resulting from inherent characteristics of a surface thereof and imbalance of particle size distribution.

### PRIOR ART LITERATURE

### Non-patent literature

Wust et al. Lancet Oncology, 2002, 3:487-497.

### SUMMARY

Aspects of the disclosure are to address at least the above-mentioned problems and/or disadvantages and to provide at least the advantages described below. Accordingly, an aspect of the disclosure is to provide a composition for treating liver cancer, the composition comprising iron oxide magnetic particles, wherein each of the iron oxide magnetic particles contains: a core including an iron oxide derived from a composite of iron and at least one type of a compound selected from a group consisting of aliphatic hydrocarbon acid salts having 4 to 25 carbon atoms and amine compounds; MXₙ; and at least one selected from a group consisting of folate, glycyrrhetinic acid, and glucose, wherein M is selected from a group consisting of Cu, Sn, Ph, Mn, Ir, Pt, Rh, ReAg, Au, Pd and Os, wherein X is selected from a group consisting of F, Cl, Br and I, wherein n is an integer of 1 to 6, wherein the iron oxide magnetic particle has an average particle diameter of onm to 20 nm.

Another aspect of the disclosure is to provide a hepatocyte targeting carrier comprising the iron oxide magnetic particles.

Hereinafter, various embodiments of the disclosure are described. The disclosure is not limited to specific embodiments. Various modifications, equivalents and/or alternatives of the embodiments of the disclosure are included in the disclosure. In connection with the description of the drawings, like reference numerals may be used for like components.

The terminology used herein is for the purpose of describing particular embodiments only and is not intended to limit the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof.

As used herein, the term “and/or” includes any and all combinations of one or more of the associated listed items. Expression such as “at least one of” when preceding a list of elements may modify the entirety of list of elements and may not modify the individual elements of the list. When referring to “C to D”, this means C inclusive to D inclusive unless otherwise specified.

The expression “configured to” as used herein may be used interchangeably with, for example, “suitable for”, “having the ability to”, “designed to”, “adapted to”, “made to”, or “capable of”, depending on the context. The term “configured to” does not necessarily mean only “specifically designed to”.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The embodiments disclosed herein is presented for description and understanding of the disclosed technical content, and does not limit the scope of the disclosure. Accordingly, the scope of the disclosure should be construed as including all changes or various other embodiments based on the technical spirit of the disclosure.

Hereinafter, a preferred embodiment of the disclosure will be described in detail. The terms or words used in the present specification and claims should not be construed as being limited to their ordinary or dictionary meanings. The terms or words used in the present specification and claims should be interpreted as a meaning and concept consistent with the technical idea of the disclosure based on the principle that the inventor may adequately define the concept of a term in order to best describe his/her invention.

Therefore, the configuration of the embodiments described in this specification is only some of the most preferable embodiments of the disclosure and does not represent all the technical ideas of the disclosure. Thus, it should be understood that various equivalents thereto and modifications thereof may be present at the time of filing the present application.

In one aspect of the present disclosure, the term “about” is intended to include errors in a preparation process included in specific values or slight numerical adjustments that fall within the scope of the technical spirit of the present disclosure. For example, the term “about” means a range of ±10%, in one aspect, ±5%, in another aspect, ±2% of a value which the term modifies.

Hereinafter, the disclosure will be described in detail.

One aspect of the disclosure provides a composition for treating liver cancer, the composition comprising iron oxide magnetic particles, wherein each of the iron oxide magnetic particles contains: a core including an iron oxide derived from a composite of iron and at least one type of a compound selected from a group consisting of aliphatic hydrocarbon acid salts having 4 to 25 carbon atoms and amine compounds; MXₙ; and at least one selected from a group consisting of folate, glycyrrhetinic acid, and glucose, wherein M is selected from a group consisting of Cu, Sn, Pb, Mn, Ir, Pt, Rh, Re, Ag, Au, Pd and Os, wherein X is selected from a group consisting of F, Cl, Br and I, wherein n is an integer of 1 to 6, wherein the iron oxide magnetic particle has an average particle diameter of 6 nm to 20 nm.

The core is specifically an iron oxide core, and includes iron oxide derived from the composite, The “iron oxide” is an oxide of iron. For example, the iron oxide includes at least one selected from a group consisting of Fe₁₃O₁₉, Fe₃O₄(magnetite), γ-Fe₂O₃(maghemite), α-Fe₂O₃(hematite), β-Fe₂O₃(beta phase), ε-Fe₂O₃(epsilon phase), FeO (Wstite), FeO₂(iron dioxide), Fe₄O₅, Fe₅O₆, Fe₅O₇, Fe₂₅O₃₂, Ferrite type and delafossite. The disclosure is not limited thereto.

The term “heavy atom” may include atoms heavier than B (boron) and may include, for example, Mn, Co, Cu, Se, Sr, Mo, Ru, Rh, Pd, Ag, Cd, Sn, Ba, Ta. W Re, Os, Ir, Pt, Au, Hg, Tl, Pb. In the iron oxide m.agrietic particles of the disclosure, a bond between the iron oxide particles and the heavy atom-halogen compound and a bond between the heavy atom-halogen are very stable, so that each component, i.e., each of iron oxide, heavy atom, and halogen element may not cause harm to the human body.

The MXₙ may include at least one selected from a group consisting of CuF, CuF₂, CuF₃, CuCl, CuCl₂, CuBr, CuBr₂, CuI, CuI₂ and CuI₃. Preferably, MXₙ may include at least one selected from a group consisting of CuF, CuCl, CuBr and CuI. In one embodiment, MXₙ may be CuI.

The meaning that MXₙ is contained in the iron oxide magnetic particle may mean that a physical or chemical bond is formed between the core surface or the iron oxide particle and MXₙ. Specifically, MXₙ may be disposed between the iron oxide particles. The iron oxide and MXₙ may be bonded to each other via hydrogen bonding. The MXₙ is formed by introducing MXₙ on the surface of the iron oxide core using a general coating method or by introducing MXₙ thereon using a doping method such as a diffusion process or an ion implantation process. Alternatively, iron oxide crystal nuclei may be formed inside MXₙ such that MXₙ acts as a shell structure. Preferably, the core of the iron oxide magnetic particles may be doped with MXₙ.

Because MXₙ is present around the iron oxide particle, the iron oxide magnetic particles may have magnetism, and may amplify the contrast effect of the iron oxide under relatively low alternating magnetic field intensity and/or low frequency magnetic field or various radiation conditions.

In one embodiment, the iron oxide may be derived from the composite of iron and at least one type of a compound selected from a group consisting of an aliphatic hydrocarbon acid salt having 4 to 25 carbon atoms and an amine-based compound. Examples of the aliphatic hydrocarbon acid salt having 4 to 25 carbon atoms may include at least one selected from a group consisting of butyrate, valerate, caproate, enanthate, caprylic acid, pelargonate, caprate, laurate, myristate, pentadecylate, acetate, palmitate, palmitoleate, margarate, stearate, oleic acid salt, bacinate, linoleate, (9,12,15)-linoleate, (6,9,12)-linolenate, eleostearate, tuberculin stearate, racidate, arachidonic acid salt, behenate, lignocerate, nerbonate, ceretate, tnontanate, melisate and a peptide salt including one or more amino acids. These compounds may be used alone or in a form of a mixed acid salt of two or more thereof.

A metal element constituting the aliphatic hydrocarbon acid salt having 4 to 25 carbon atoms may include at least one selected from a group consisting of calcium, sodium, potassium and magnesium.

Examples of the amine-based compound may include at least one selected from a group consisting of methylamine, ethylamine, propylamine, isopropylamine, butylamine, amylamine, hexylamine, octylamine, 2-ethylhexylamine, nonylamine, decylamine, lauryl amine, pentadecylamine, cetylamine, stearylamine and cyclohexylamine, dimethylamine, diethylamine, dipropylamine, diisopropylamine, dibutylamine, diamylamine, dioctylamine, di(2-ethythexyl)amine, didecylamine, dilaurylamine, dicetylamine, distearylamine, methylstearylamine, ethylstearylamine and butylstearylamine, triethylamine, triamylamine, trihexylamine and trioctylamine, triallylamine, oleylamine, laurylaniline, stearylaniline, triphenylamine, N,N-dimethylaniline and dimethylbenzylaniline, monoethanolamine, diethanolamine, triethanolamine, dimethylaminoethanol, diethylenetriamine, triethylenetetramine, tetraethylenepentaamine, benzylamine, diethylaminopropylamine, xylylenediamine, ethylenediamine, hexamethylenediamine, dodecamethylenediamine, dimethylethylenediamine, triethylenediamine, guanidine, diphenylguanidine, N,N,N′,N′-tetramethyl-1,3-butanediamine, N,N,N′,N′-tetramethylethylenediamine, 2,4,6-tris(dimethylaminomethyl)phenol, morpholine, N-methylmorpholine, 2-ethyl-4-methylimidazole and 1,8-diazabicyclo (5,4,0)undecene-7 (DBU).

In one embodiment, the composite may be an iron-oleic acid composite.

The X may include a radioactive isotope of X or a mixture of radioactive isotopes of X. The term “radioisotope” refers to any compound in which one of two or more atoms having the same atomic number is replaced with another having an atomic mass or mass number different from an atomic mass or mass number normally found in nature. Examples of isotopes suitable for being included in the compound of the disclosure may include, for example, isotope of fluorine such as ¹⁸F, isotope of chlorine such as ³⁶Cl, isotopes of bromine such as ⁷⁵Br, ⁷⁶Br, ⁷⁷Br and ⁸²Br, and isotopes of iodine such as ¹²³I, ¹²⁴I, ¹²⁵I and ¹³¹I alone or in a mixed manner.

The iron oxide magnetic particle may have an average particle diameter (d50) of 6 nm to 20 nm. The average particle diameter may be in a range of 6 nm to 15 nm, 8 nm to 15 nm, or 8 nm to 12 nm. When the average particle diameter of iron oxide magnetic particles is smaller than 6 nm, the particle may be excreted directly to the kidneys and may not be accumulated in the liver to treat liver cancer. When the average particle diameter of the particles exceeds 20 nm, they may accumulate in organs other than the liver or induce an immune response, and the excretion thereof may be too slow, which may cause toxicity. In the average particle diameter range of the iron oxide magnetic particles as defined above, the particle may be captured by Kupffer cells, which are macrophages in the liver, so that they may stay in the liver while forming a composite with the protein. Thus, when the diameter is smaller than the above range, they are excreted through capillaries.

In the iron oxide magnetic particle, MXₙ may be contained at about 1 to 13 mol %, preferably about 1 to 8 mol %, more preferably about 3 to 8 mol %, based on 100 mol % of the composite composed of the iron and at least one type of a compound selected from a group consisting of an aliphatic hydrocarbon acid salt having 4 to 25 carbon atoms and an amine-based compound.

In the iron oxide magnetic particle, MXₙ may be contained at a weight ratio 1:0.005 to 0.08, and preferably 1:0.008 to 0.08 based on the iron oxide included in the particle. The ratio may be measured using an inductively coupled plasma (ICP) mass spectroscopy which is a metal content analysis equipment. When MXₙ is contained in the iron oxide magnetic particle at the content thereof within the above range, the particle may secure an excellent specific loss power, and may secure a high temperature change under an external alternating magnetic field or When irradiated with radiation.

In one embodiment, in the iron oxide magnetic particle, a hydrophilic or charged ligand or polymer may be coated on at least a portion of the surface of the iron oxide particle core. The hydrophilic ligand may be introduced to increase solubility in water and stabilization of the iron oxide magnetic particles according to an embodiment, or to enhance targeting toward or penetration into specific cells such as cancer cells. The hydrophilic ligand may preferably have biocompatibility, and may include, for example, at least one selected from a group consisting of polyethylene glycol, polyethyleneamine, polyethyleneimine, polyactylic acid, polymaleic anhydride, polyvinyl alcohol, polyvinylpyrrolidone, polyvinylamine, polya.crylamide, polyethylene glycol, phosphoric acid-polyethylene glycol, polybutylene terephthalate, polylactic acid, polytrimethylene carbonate, polydioxanone, polypropylene oxide, polyhydroxyethyl methacrylate, starch, dextran derivatives, sulfonic acid amino acid, sulfonic acid peptide, silica and polypeptides. The disclosure is not limited thereto. Preferably, the hydrophilic ligand may be a phosphoric acid-polyethylene glycol-based material. Specifically, the hydrophilic ligand may be phosphoethanolamine-polyethylene glycol such as 1,2-disteroyl-sn-glycero-3-phosphoethanolamine-N-methoxy(polyethylene glycol), or 1,2-disteroyl-sn-glycero-3-phosphoethanolamine-N-(polyethylene glycol).

The iron oxide magnetic particle may contain at least one selected from a group consisting of folate, glycyrrhetinic acid, and glucose. The folate, glycyrrhetinic acid, or glucose may function as a targeting agent to help deliver the particle to a specific target organ or target cell. More specifically, it may be preferable that the iron oxide magnetic particle contains glycyrrhetinic acid. For example, the iron oxide magnetic particle may contain a glycyrrhetinic acid alone; a combination of glycyrrhetinic acid and folate; a combination of glycyrrhetinic acid and glucose; or a combination of glycyrrhetinic acid, folate, and glucose.

At least one selected from a group consisting of folate, glycyrrhetinic acid, and glucose may be bound to the hydrophilic ligand. Thus, the particle may contain hydrophilic ligand-folate, hydrophilic ligand-glycyrrhetinic acid, or hydrophilic ligand-glucose. For example, when the hydrophilic ligand is a phosphoric acid-polyethylene glycol-based material, the iron oxide magnetic particle may contain 1,2-disteroyl-sn-glycero-3-phosphoethanolamine -N-(polyethylene glycol)-folate, 1,2-disteroyi-sn-glycero-3-phosphoethanolamine -N-(polyethylene glycol)-glycyrrhetinic acid, or 1,2-disteroyl-sn-glycero-3-phosphoethanolamine -N-(oolyethylene glycol)-glucose.

A weight ratio of the hydrophilic ligand to the targeting material, that is, one selected from folate, glycyrrhetinic acid, and glucose is in range of 15 to 5:1, 12 to 8:1, 10 to 8:1, or 9:1. When the weight ratio of the hydrophilic ligand to the targeting material exceeds or falls below the above weight ratio, the effect of increasing the magnetic drug delivery resulting from folate, glycyrrhetinic acid, or glucose may be reduced.

One selected from the hydrophilic ligand-folate, the hydrophilic ligand-glycyrrhetinic acid, and the hydrophilic ligand-glucose may have a density of 5 to 15, 5 to 12, 5 to 10, or 7 to 9 per 1 nm² of a core particle surface area. When the density is smaller than the above defined range, the solubility in water of the iron oxide magnetic particles decreases, and thus the delivery efficiency may decrease, or there may be a risk of thrombus formation, edema, pain, etc. When the density exceeds the above range, the size of the iron oxide magnetic particle becomes too large, or magnetism may decrease.

The composition for treating liver cancer may further contain a pharmaceutically acceptable carrier depending on the administration method, administration location, and organ to be diagnosed. The composition for the treatment of liver cancer may be administered in intravenous injection, subcutaneous injection, intramuscular injection, intraperitoneal injection, intralesional injection, intratumoral injection, etc. The composition may be preferably administered in intravenous administration. When the composition for treatment of liver cancer is administered intravenously, the composition may be formulated into an aqueous solution or suspension using a commonly known solvent such as isotonic sodium chloride solution, Hank's solution, or Ringer's solution.

The composition for treating liver cancer may be used in combination with external stimuli such as radiation, magnetic field and radio waves, and may be applied to hyperthermia. The iron oxide magnetic particles included in the composition for the treatment of liver cancer may be applied to hyperthermia because the particles may secure high specific loss power while having high reactivity to external stimuli such as radiation, magnetic fields and radio waves. The term. “hyperthermia” means exposing body tissues to a temperature higher than normal body temperature to kill lesion cells including cancer cells or to make these cells more sensitive to radiation therapy or anticancer drugs.

In the heavy atom-halogen compound such as MXₙ, the permittivity and capacitance according to the type of the heavy atom and the type of the halogen may vary (the permittivity/capacitance may vary as an atomic shell increases from F to I in a periodic table). Thus, the heavy atom-halogen compound may be coupled to the iron oxide which is a magnetic substance, not only to increase the magnetic intensity, but also to increase the size or total amount of electromagnetic field energy that the compound may absorb, thereby increasing the amount of thermal energy emitted from the final iron oxide-based magnetic particles. Thus, in the electromagnetic field energy environment of not only the existing high-frequency (200 kHz, or higher) range, but also relatively low and medium-frequency (50 Hz to 200 kHz) band, higher thermal energy emission (conversion) efficiency (ILP: Intrinsic loss power) may be improved or increased compared to conventional iron oxide-based magnetic particles.

In addition, because the iron oxide magnetic particles included in the composition for treating liver cancer have a magnetic property, the composition may function as a contrast agent applicable to a diagnostic device using a magnetic property. Therefore, because the composition for treating liver cancer may be used to diagnose cancer without additional administration of a contrast agent, diagnosis and treatment of cancer may be performed at the same time. When the composition of the disclosure is used, there is no need for additional contrast medium administration, so that the risk of side effects is small and the burden on the patient is low. A diagnostic device to which the composition may be applied is not limited particularly. Because the contrast agent including the iron oxide magnetic particles has both a negative contrast agent component and a positive contrast agent component, it has a high contrast and exhibits an excellent contrast effect. In particular, the contrast agent including the iron oxide magnetic particles exhibits a higher radiation absorption HU (hounsfield unit) value and CT contrast effect than conventional iodine-based (Iohexol or Iopamidol) or gold nano-CT contrast agents exhibit. It is reported that the existing iodine-based contrast agent exhibits 3000 HU (4.6 HU based on 1 mg) based on 647 mg/ml, and gold nanoparticles exhibit about 5 to 50 HU based on 1 mg. On the other hand, the contrast medium including the iron oxide magnetic particles according to the disclosure exhibits about 50 to 100 HU based on 1 mg.

The composition according to the disclosure may be used as a CT contrast agent as well as a contrast agent for X-ray imaging, Magnetic Resonance imaging (MRI), US, optical imaging, Single Photon Emission Computed Tomography (SPECT), Positron Emission Tomography (PET), Magnetic Particle Imaging (MPI), flat panel imaging, and rigid, flexible or capsule endoscopy.

The fact that one type of the contrast agent may be used for various devices may be very useful when complex tests are required. For example, when both CT scan and MRI scan need to be performed within a short time, CT contrast agent 1 and MRI contrast agent 2 are separately injected into the body. Thus, as the different contrast agents are mixed with each other in the body, the test result may be unclear. As a subject receives a separate contrast agent for each test, the probability of causing toxicity increases. However, the contrast medium containing the iron oxide magnetic particles according to the disclosure may be used without limitation in various devices, so that this inconvenience may be reduced,

In one embodiment, when the composition for treatment of liver cancer is used for hyperthermia or diagnosis, the contrast effect may be exhibited under a magnetic field having a frequency of 1 kHz to 1 MHz or lower or having an intensity of 20 Oe (1.6 kA/m) to 200 Oe (16 kA/m) or lower. The alternating magnetic field irradiated to the subject after administering the contrast agent to the subject may have a frequency of 1 kHz to 1 MHz, or a frequency of 30 kHz to 120 kHz. In general, in order to change a spin state from a singlet to a triplet, an alternating magnetic field of 1 MHz or higher must be applied. However, when the composition according to the disclosure is used, a spin state may be changed from a singlet to a triplet even under alternating magnetic fields of tens to hundreds of kHz. In addition, the alternating magnetic field may have a magnetic field intensity of 20 Oe (1.6 kA/m) to 200 Oe (16.0 kA/m), 80 Oe (6.4 kA/m) to 160 Oe (12.7 kA/m), or 140 Oe (11.1 kA/m). The contrast agent according to one embodiment is useful in that it may be used even in an alternating magnetic field of a low magnetic field intensity and/or frequency, which is relatively harmless to the human body. This is not the case in the existing high-energy method.

The iron oxide magnetic particles included in the composition of the disclosure may be administered intravenously and then excreted in urine from the body within 2 weeks after the administration. In addition, the particles may not be decomposed at an acidity of about pH 5.5 to 6.5, and may not non-specifically bind to a protein in the body.

Another aspect of the disclosure provides a hepatocyte targeting carrier including the iron oxide magnetic particles. The hepatocyte may specifically be a liver cancer cell.

Because the iron oxide magnetic particles are specifically delivered to the liver, the particles may bind to an active ingredient and thus the active ingredient may be delivered to the hepatocyte. The meaning of “liver-specific delivery” means that 50% or more, 60% or more, 70% or more, 80% or more, or 90% or more of the AUC as measured within 24 hours after the administration accumulates in the liver, and more specifically means that there is little accumulation of the particles in the kidneys or lungs in which the blood vessels are dense. The term “little accumulation” means accumulation of smaller than 50%, smaller than 40%, smaller than 30%, smaller than 20%, or smaller than 10% of the AUC as measured within 24 hours after the administration. The active ingredient may be a nutrient beneficial to hepatocytes or a drug for treating liver disease, for example, a drug for treating diseases such as liver cancer, hepatitis, alcoholic liver disease, cirrhosis, fatty liver, and liver cirrhosis. Examples of therapeutic agents for liver cancer include, but are not limited to, sorafenib, lenvatinib, regorafenib, ramucirumab, caboxantinib, and atezolizumab.

Another aspect of the disclosure is to provide a method for preparing the above-described iron oxide magnetic particles.

Specifically, the preparation method of iron oxide magnetic particles preparing an iron oxide core including an iron oxide derived from a composite of iron and at least one type of a compound selected from a group consisting of aliphatic hydrocarbon acid salts having 4 to 25 carbon atoms and amine compounds, introducing MAₙ into the iron oxide core by mixing MAₙ with the iron oxide core and heating the mixture, and mixing BₙX with the iron oxide core into which the MAₙ has been introduced and heating the mixture, thereby forming MXₙ, wherein M is selected from a group consisting of Cu, Sri, Pb, Mn, Tr, Pt, Rh, Re, Ag. Au, Pd and Os. wherein each of A and X is independently selected from a group consisting of F, Cl, Br and I, wherein B is selected from a group consisting of Li, Na, and K, and n is an integer of 1 to 6. The step of forming MXₙ may include further adding the hydrophilic ligand and at least one selected from a group consisting of folate, glycyrrhetinic acid, and glucose thereto.

The step of preparing the core may include reacting an iron halogen salt, and at least one type of a compound selected from a group consisting of aliphatic hydrocarbon acid salts having 4 to 25 carbon atoms and amine compounds with each other under presence of water, thereby forming the iron oxide core; and separating the iron oxide core.

The iron halogen salt is a salt composed of iron and a halogen element, and may include, for example, ferrous chloride (FeCl₂), ferric chloride (FeCl₃), etc., but is not limited thereto.

More specifically, the step of forming the iron oxide core may include the reaction in a solution that is a mixture of an organic solvent and water. The organic solvent may be methanol, ethanol, propanol, butanol, hexane, chloroform, acetone, acetic acid, or a. mixture thereof, but is not limited thereto. The reaction may occur at 40° C. to 100° C., 40° C. to 80° C., or 40° C. to 60° C. for 3 hours to 6 hours or more. The separating may include separating an organic layer including the iron oxide core as a reaction product. The reaction may be repeated at least two times.

The separating of the iron oxide core may further include a step of evaporating the organic solvent at 100° C. to 120° C.

The step of introducing MAₙ into the iron oxide core in the preparation method may include reaction for 20 minutes to 40 minutes at a high temperature of 300° C. to 350° C. under nitrogen gas. In order to separate the iron oxide core into which the MAₙ has been introduced, the method may further include a step of mixing the cores with a solution that is a 2:1 mixture of ethanol and hexane and centrifuging the cores.

In the step of forming MXₙ in the preparation method, the element A in the iron oxide core into which MAₙ has been introduced is substituted with X. Because the preparation method according to the disclosure employs an ion exchange method rather than a method of directly introducing MXₙ into the core, the doping efficiency of MXₙ is high, so that the preparation efficiency is high, and the iron oxide nanoparticles having uniform and high magnetism may be prepared.

In the step of forming MXₙ, a hydrophilic ligand and at least one selected from a group consisting of folate, glycyrrhetinic acid, and glucose may be additionally mixed therewith. In this process, as MXₙ is formed, a hydrophilic ligand and at least one selected from a group consisting of folate, glycyrrhetinic acid, and glucose may be additionally introduced into the iron oxide core to constitute the iron oxide magnetic particle. The step of forming MXₙ may further include applying microwaves, heating, sonication, filtering, centrifugation, etc. to increase the ion exchange efficiency and to make the sizes of the iron oxide magnetic particles uniform.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic diagram of a preparation process and a structure of a nanoparticle according to an embodiment of the disclosure;

FIG. 2 is a graph showing uptake efficiency of nanoparticles into liver cancer cells according to an embodiment of the disclosure;

FIG. 3 is a graph showing biotoxicity test results against liver and kidney of nanoparticles according to an embodiment of the disclosure;

FIG. 4 is a graph showing biodistribution in an animal model of nanoparticles according to an embodiment of the disclosure;

FIG. 5 is a graph showing delivery efficiency of nanoparticles according to an embodiment of the disclosure to liver cancer cells; and

FIG. 6 is a graph showing liver cancer treatment effect of nanoparticles according to an embodiment of the disclosure,

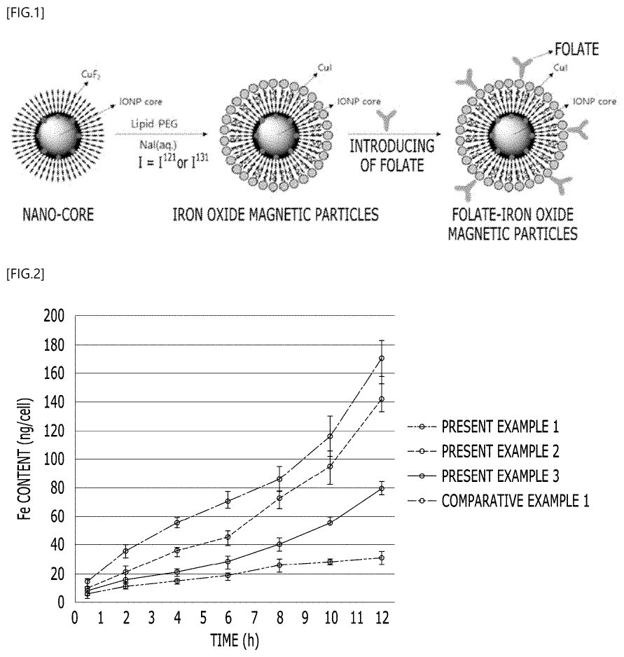

### DETAILED DESCRIPTION

Hereinafter, in order to help understanding of the disclosure, Example will be described in detail. However, Examples according to the disclosure may be changed into various other forms, and the scope of the disclosure should not be construed as being limited to the following Examples. Examples of the disclosure are provided to more fully explain the disclosure to a person with average knowledge in the art.

Present Example 1: Preparation of Iron Oxide Magnetic Particles Into Which GA (glycyrrhetinic acid) Is Introduced

(a) Formation of iron-oleic acid or iron-oleyl amine composite

FeCl₃.6H₂O 6.218 g (60 mmol), sodium oleate 54.79 g (180 mmol), hexane 224 ml, ethanol 120 ml, and deionized water 90 ml reacted with each other at 50° C. for about 4 hours at 900 rpm while vigorously stirring the mixture. After the reaction solution was cooled to room temperature, a transparent lower layer was removed using a separatory funnel. 100 ml of water was mixed with a brown upper organic layer, and the mixture was shaken, and a lower water layer was removed again. This was repeated 3 times. A remaining brown organic layer was transferred to a beaker which was heated at 110° C. overnight to evaporate hexane therefrom, thereby obtaining iron-oleic acid composite as iron oxide core particles.

(b) Synthesis of iron oxide magnetic particles containing CuF₂

The iron-oleic acid composite as prepared above 4.5 g (5 mmol) and oleic acid 0.8 ml (2.5 mmol) were mixed with each other, and CuF₂30.5 mg (0.3 mmol) and 1-octadecene 15 ml were added thereto and mixed therewith. The mixture was placed in a round bottom flask and heated to 90° C. in a vacuum for 30 minutes to remove gas and moisture therefrom. Nitrogen was injected thereto and a temperature was raised to 200° C. Thereafter, the temperature was raised to 320° C. at a rate of 3.3° C./renin and then reaction occurred for 30 minutes. After cooling the reaction solution, the cooled solution was transferred to a 50 ml conical tube, and then 30 ml of ethanol and hexane were injected thereto in a 2:1 ratio, and then centrifugation was carded out to precipitate the particles. The precipitated particles were washed with 25 ml of ethanol and 15 ml of hexane, and the resulting precipitate was dispersed in hexane. Then, the dispersion was dispensed into a 50 ml vial. The solvent was evaporated therefrom, and then a resulting product was redispersed in toluene such that the iron oxide had a concentration of 25 mg/ml.

(c) Introduction of 1 and glycyrrhetinic acid to iron oxide magnetic particles containing CuF₂

10 mg of iron oxide magnetic particles containing CuF₂ were dispersed in 1 mL of chloroform. The dispersion, 2 mL of deionized water, 20 mg of NaI, and DSPE-PEG2000 (1,2-distearoyl-sn-glycero-3-phosphoethanolamine-N-[methoxy(polyethylene glycol)]-2000), and DSPE-PEG2000-Glycyrrhetinic acid (1,2-distearoyl-sn-glycero-3-phosphoethariolamine -N-(polyethylene glycol)-glycyrrhetinic acid) at a density of 8 per a particle surface area. (1 nm²) and in a weight ratio of 9:1 were put into a 50 mL vial. For one minute, the vial was subjected to an operation of a microwave 2.4 GHz 1000W.

After removing the solution using an evaporator, 3 ml of deionized water was added thereto and a resulting solution was dispersed via sonication for 5 minutes. After the dispersing, the dispersion, and ethanol and deionized water in a ratio of 2:8 were input to Amicon 100k and centrifugation was carried out (5,000 rpm, 5 m). Deionized water was input to Amicon 100k and centrifugation (5,000 rpm, 5 m) was conducted to obtain iron oxide nanoparticles. An average particle diameter of the prepared nanoparticies was 10 nm.

Present Example 2: Preparation of Iron Oxide Magnetic Particles To Which Folate Is Introduced

A process was performed in the same manner as in Present Example 1, except that a step of introducing I and folate to the iron oxide magnetic particles containing CuF₂ of Present Example 1-(c), and subsequent steps were performed as follows.

10 mg of iron oxide magnetic particles containing CuF₂ were dispersed in of chloroform. The dispersion, 2 mL of deionized water, 20 mg of NaI, and DSPE-PEG2000 (1,2-distearoyl-sn-glycero-3-phosphoethanolamine-N-[methoxy(polyethylene glycol)]-2000) and DSPE-PEG2000-Folate (1,2-distearoyl-sn-glycero-3-phosphoethanolarnine-N-(polyethylene glycol)-folate) at a density of 8 per a particle surface area (1nm²) and in a weight ratio of 9:1 were put into a 50 mL vial. For one minute, the vial was subjected to an operation of a microwave 2.4 GHz 1000W, A subsequent procedure was performed in the same manner as in Present Example 1. The average particle diameter of the prepared nanoparticles was 10 nm.

Present Example 3: Preparation Of Iron Oxide Magnetic Particles To Which Glu (Glucose) Is Introduced

A process was performed in the same manner as in Present Example 1, except that a step of introducing I and glucose to the iron oxide magnetic particles containing CuF₂ of Present Example 1-(c), and subsequent steps were performed as follows,

10 mg of iron oxide magnetic particles containing CuF₂ were dispersed in 1 mL of chloroform. The dispersion, 2 mL of deionized water, 20 mg of NaI, and DSPE-PEG2000 (1,2-distearoyl-sn-glycero-3-phosphoethanolamine-N-knethoxy(polyethylene glycol)-2000), and DSPE-PEG2000-glucose (1,2-distearoyl-sn-glycero-3-phosphoethanolamine-N-(polyethylene glycol)-glucose) at a density of 8 per a particle surface area (1 nm²) and in a weight ratio of 9:1 were put into a 50 mL, vial. For one minute, the vial was subjected to an operation of a microwave 2.4 GHz 1000W. A subsequent procedure was performed in the same manner as in Present Example 1. The average particle diameter of the prepared nanoparticles was 10 nm.

Present Example 4: Preparation Of Iron Oxide Magnetic Particles To Which GA And ¹³¹I Are Introduced

A process was performed in the same manner as in Present Example 1, except that a step of introducing GA and ¹³¹I to the iron oxide magnetic particles containing CuF₂ of Present Example 1-(c), and subsequent steps were performed as follows.

10 mg of iron oxide magnetic particles containing CuF₂ were dispersed in 1 mL of chloroform. The dispersion, 2 mL of deionized water, 1 mL of NaI¹³¹ (185 MBq(5mCi)), DSPE-PEG2000(1,2-distearoyl-sn-glycero-3-phosphoethanolamine-N-[methoxy(polyethylene glycol)]-2000), and DSPE-PEG2000-glycyrrhetinic acid) (1,2-distearoyl-sn-alycero-3-phosphoethanolamine-N-(polyethylene glycol)-glycyrrhetinic acid)) at a density of 8 per a particle surface area (1 nm²) and in a weight ratio of 9:1 were put into a 50 mL vial. For one minute, the vial was subjected to an operation of a microwave 24 GHz 1000W. A subsequent procedure was performed in the same manner as in Present Example 1.

The average particle diameter of the prepared nanoparticles was 10 nm. When the iron oxide magnetic particles to which GA and ¹³¹I were introduced as prepared in the above experiment had a radiation dose of 50 MBq (1.35 mCi) as measured with a gamma-counter.

Comparative Example 1: Preparation of Iron Oxide Magnetic Particles to which Only I is Introduced

A process was performed in the same manner as in Present Example 1, except that a step of introducing I to the iron oxide magnetic particles containing CuF₂ of Present Example 1-(c), and subsequent steps were performed as follows.

10 mg of iron oxide magnetic particles containing CuF₂ were dispersed in 1 mL of chloroform. The dispersion, 2 mL of deionized water, 1 ml of NaI, and SSPE-PEG2000 (1,2-distearoyl-sn-glycero-3-phosphoethanolamine-N-[methoxy(polyethylene glycol)]-2000) at a density of 8 per a particle surface area (1 nm²) were put into a 50 mL vial. For one minute, the vial was subjected to an operation of a microwave 2.4 GHz 1000W. A subsequent procedure was performed in the same manner as in Present Example 1.

Comparative Example 2: Preparation of Iron Oxide Magnetic Particles to which Only ¹³¹I is Introduced

A process was performed in the same manner as in Present Example 1, except that a step of introducing ¹³¹I to the iron oxide magnetic particles containing CuF₂ of Present Example 1-(c), and subsequent steps were performed as follows.

10 mg of iron oxide magnetic particles containing CuF₂ were dispersed in 1 mL of chloroform. The dispersion, 2 mL of deionized water, linL of NaI¹³¹ (185MBq(5mCi)), and DSPE-PEG2000 (1,2-distearoyl-sn-glycero-3-phosphoethanolamine-N-[methoxy(polyethylene glycol)]-2000) at a density of 8 per a particle surface area (1 nm²) were put into a 50 mL vial. For one minute, the vial was subjected to an operation of a microwave 2.4 GHz 1000W. A subsequent procedure was performed in the same manner as in Present Example 1.

Test Example 1: Uptake Test Into Live Cancer Cells (in vitro)

The uptake of the iron oxide magnetic particles according to the disclosure into the liver cancer cells was examined to evaluate an ability of the particle to deliver the active ingredient to liver cancer cells. Specifically, hepG2 cells, which are liver cancer cells, were treated with the iron oxide magnetic particles at 200 μug/mL. Then, extracellular iron oxide magnetic particles were removed at each timing, and then the cells were decomposed with an acidic solution and 4% potassium ferrocyanide solution was added thereto. Then, a delivery rate of the iron ions into the hepatocarcinoma cells over time was measured based on UV absorbance value using Prussian blue staining.

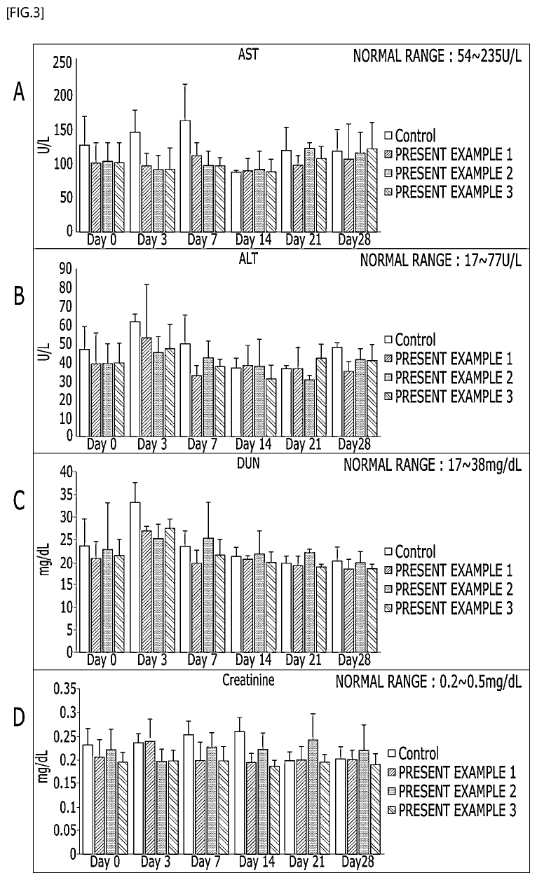

The results are shown in FIG. 2. Present Examples 1 to 3 exhibited a higher delivery rate into hepatocellular carcinoma, compared to Comparative Example 1. Among Present Examples 1 to 3, Present Example 1 exhibited the highest delivery rate into hepatocellular carcinoma.

Test Example 2: In Vivo Toxicity Test

It was tested Whether the iron oxide magnetic particles according to the disclosure have biotoxicity. Specifically, after administering each of Present Examples 1 to 3 to 100 mg/kg to Balb/c nude mice, orbital blood was collected for liver and kidney enzymes before the administration and on 1st, 7th, 14th, and 28th days after the administration. Then, blood biochemical levels were tested. In a control group, only water for injection as used for administering Present Examples 1 to 3 thereto was administered thereto.

The results are shown in FIG. 3. Based on a result of the test, it was identified that for all of Present Examples 1 to 3, enzyme levels related to liver and kidney among all observed organs were within a normal range.

Test Example 3: Biodistribution Test in Animal Model

Animal experiments were conducted to evaluate delivery effect of the iron oxide magnetic particles according to the disclosure into the liver. Specifically, after 100 mg/kg of Presort Example I was administered to Balb/c nude mice at the tail vein thereof, the distribution of the particles in each organ in the body and the change thereof over time were identified based on iron ion analysis via hourly ICP-MS analysis.

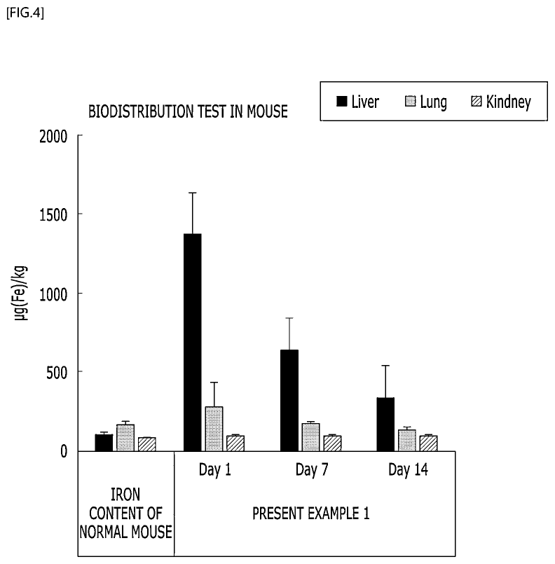

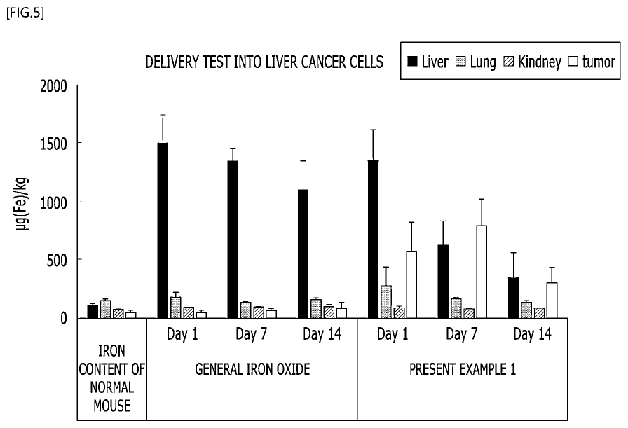

The results are shown in FIG. 4. Based on a result of observing distribution of Present Example 1 in each organ tissue, it was identified that the iron oxide magnetic particles were specifically delivered only to the liver, but were hardly delivered to the kidneys or lungs, and almost all of the iron oxide magnetic particles delivered to the liver were excreted within about 2 weeks.

Test Example 4: Delivery Test Into Liver Cancer Cell

Animal experiments were conducted to evaluate the delivery effect of iron oxide magnetic particles according to the disclosure into the liver cancer cells. The animal model as used was a xenograft mouse model, which was produced by transplanting human liver cancer cells into buttocks of Balblc nude mice to induce the liver cancer. After administering Present Example 1 and general iron oxides as a control at 100 mg/kg to the tail vein of the produced xenograft mouse model, the delivery rate to the liver cancer cells, the distribution in the body and change thereof over time were identified based on iron ion analysis via ICP-MS analysis based on timings.

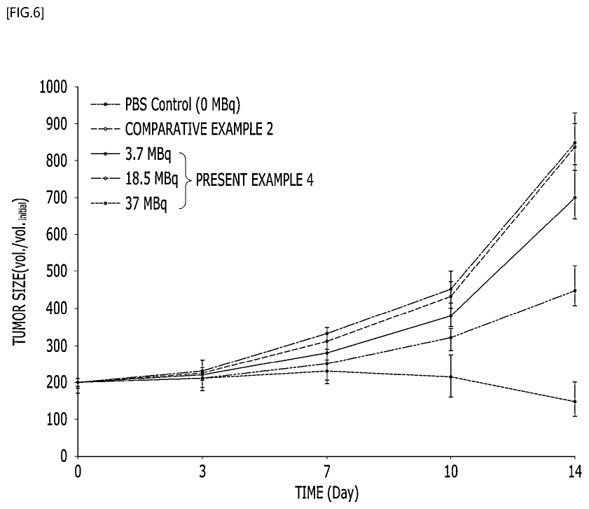

The results are shown in FIG. 5. Based on a result of observing the distribution of Present Example 1 in each organ tissue, it was identified that the iron oxide magnetic particles initially delivered to the liver were accumulated into liver cancer cells over time, and the maximum amount had been accumulated in the liver cancer cells after 1 week, and almost all thereof were excreted after about 2 weeks. Further, it was identified that the particles were hardly delivered to the kidneys or lungs. On the other hand, it was observed that it the conventional iron oxide particle was not transferred to the liver cancer cells after about 2 weeks, and most thereof were accumulated in the liver and were not excreted.

Test Example 5: Liver Cancer Treatment Test

An animal experiment was conducted to evaluate the liver cancer treatment effect of the iron oxide magnetic particles according to the disclosure. Specifically, we induced the liver cancer in Balblc nude mice. The liver cancer treatment effect of each of Present Example 4 (with GA) as a magnetic drug carrier which contained GA and was doped with I¹³¹, and Comparative Example 2 (w/o GA) as a magnetic drug carrier which was free of GA and was doped with I¹³¹ was identified. Further, the liver cancer treatment effect of Present Example 4 was identified based on a varying radiation dose of I¹³¹.

The results are shown in FIG. 6. Based on a result of measuring a tumor size at intervals of 3, 7, 10, and 14 days, it was identified that the liver cancer treatment effect of Present Example 4 (with GA) as a magnetic drug carrier which contained GA and was doped with was higher than that of each of the PBS control and Comparative Example 2. Further, the liver cancer treatment effect was higher at a larger radiation dose.

According to the disclosure, the composition comprising the nanoparticles according to one embodiment is delivered specifically to the liver, the composition may act on the liver cancer cells without damaging other organs.

Further, the nanoparticles according to one embodiment remain in the body for a certain period of time and are excreted outside the body within a few weeks. Thus, there is little risk of side effects such as organ damage caused by the accumulated iron oxide.

Further, nanoparticles according to one embodiment include the iron oxide magnetic particles, and thus have high responsiveness to external stimuli such as radiation, magnetic field and radio waves, and thus may be used for hyperthermia.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A composition for treating liver cancer, the composition comprising iron oxide magnetic particles, wherein each of the iron oxide magnetic particles contains:
a core including an iron oxide derived from a composite of iron and at least one type of a. compound selected from a group consisting of an aliphatic hydrocarbon acid salt having 4 to 25 carbon atoms and an amine compound;
MXₙ; and
at least one selected from a group consisting of folate, glycyrrhetinic acid, and glucose,
wherein M is selected from a group consisting of Cu, Sn, Pb, Mn, Ir, Pt, Rh, Re, Ag, Au, Pd and Os,
wherein X is selected from a group consisting of F, Cl, Br and I,
wherein n is an integer of 1 to 6,
wherein the iron oxide magnetic particle has an average particle diameter of 6 nm to 20 nm.

2. The composition of claim 1, wherein the iron oxide includes at least one selected from a group consisting of Fe₁₃O₁₉, Fe₃O₄ (magnetite), γ-Fe₂O₃ (maghemite), α-Fe₂O₃ (hematite), β-Fe₂O₃ (beta phase), ε-Fe₂O₃ (epsilon phase), FeO (Wstite), FeO₂ (iron dioxide). Fe₄O₅, Fe₅O₆, Fe₅O₇, Fe₂₅O₃₂, Ferrite type and delafossite.

3. The composition of claim 1, wherein X includes a radioactive isotope of X or a mixture of radioactive isotopes of X.

4. The composition of claim 1, wherein the composite is an iron-oleic acid composite.

5. The composition of claim 1, wherein MXₙ is CuI.

6. The composition of claim 1, wherein the iron oxide magnetic particle includes the glycyrrhetinic acid.

7. The composition of claim 1, wherein the core is coated with a hydrophilic ligand.

8. The composition of claim 1, wherein MXₙ is contained in an amount of 1 to 13 mol % based on 100 mol % of the composite.

9. The composition of claim 1, wherein the composition is used in a magnetic field having. a low frequency of 1 kHz to 1 MHz and an intensity of 20 Oe (1.6 kA/m) to 200 Oe (16.0 kA/m).

10. The composition of claim 7, wherein the hydrophilic ligand includes at least one from a group consisting of polyethylene glycol, polyethyleneamine, polyethyleneimine, polyactylic acid, polymaleic anhydride, polyvinyl alcohol, polyvinylpyrrolidone, polyvinylamine, polyacrylamide, polyethylene glycol, phosphoric acid-polyethylene glycol, polybutylene terephthalate, polylactic acid, polytrimethylene carbonate, polydioxanone, polypropylene oxide, polyhydroxyethyl methacrylate, starch, dextran derivatives, sulfonic amino acids, sulfonic acid peptide, silica and polypeptide.

11. A hepatocyte targeting carrier comprising iron oxide magnetic particles, wherein each of the iron oxide magnetic particles contains:
a core including an iron oxide derived from a composite of iron and at least one type of a compound selected from a group consisting of an aliphatic hydrocarbon acid salt having 4 to 25 carbon atoms and an amine compound;
MXₙ; and
at least one selected from a group consisting of folate, glycyrrhetinic acid, and glucose,
wherein M is selected from a group consisting of Cu, Sn, Pb, Mn, tr, Pt, Rh, Re, Au, Pd and Os,
wherein X is selected from a group consisting of F, Cl, Br and I,
wherein n is an integer of 1 to 6,
wherein the iron oxide magnetic particle has an average particle diameter of 6 nm to 20 nm.

12. The hepatocyte targeting carrier of claim 11, wherein the hepatocyte targeting carrier further contains an active iruzredient for treatment of liver cancer.

13. A method for treating liver cancer, comprising administering the composition of claim 1 to a subject in need thereof.
